(19) United States
(12) Patent Application Publication
(10) Pub. No.: US 2023/0330231 A1
(43) Pub. Date: Oct. 19, 2023

(54) Process for cure of MI's and strokes through drug induced biophysical methods.

(71) Applicant: Debasish Banerjee, Kolkata (IN)

(72) Inventor: Debasish Banerjee, Kolkata (IN)

(21) Appl. No.: 17/914,345

(22) PCT Filed: Jul. 12, 2021
(86) PCT No.: PCT/IB2021/056229

§ 371 Date: Sep. 25, 2022

(51) Int. Cl. A61K 45/06

(52) U.S. Cl. CPC A61K 45/06

(57) ABSTRACT

This invention describes a biophysical method in which combination of drugs are used for the cure of MI's and ischemic strokes, even in cases of multiple site blockages. The method is more convenient in the sense it does not require the use of specialized personnel or equipment and the combination of drugs used is more effective and safer than the existing methods. It has had a 100% success rate in preventing fatalities. It reperfuses the myocardium or cerebral tissue very rapidly and reduces haemorrhage volume in haemorrhagic strokes.

### CROSS REFERENCE TO RELATED APPLICATIONS

International Application Number: PCT/IB2021/056229 dated Jul. 12, 2021

### STATEMENT REGARDING FEDERALLY SPONSORED RESEARCH OR DEVELOPMENT

Not applicable

### THE NAMES OF PARTIES TOA JOINT RESEARCH AGREEMENT

Not applicable

### SEQUENCE LISTING

Not applicable

### STATEMENT REGARDING PRIOR DISCLOSURES BY INVENTOR OR JOINT INVENTOR

Not Applicable

### BACKGROUND OF THE INVENTION

### Patent Classification Number

This process provides a novel method for reperfusion in patients suffering from MI's, pulmonary embolism and ischemic strokes. It is speedy, and removes thrombus and emboli providing almost immediate reperfusion thereby minimizing the infarct area. In cases where there are several infarcts all are removed simultaneously, the area reperfused, and repeat attacks are rare. Earlier the agents used for thrombolysis, streptokinase, alteplase, reteplase and others, had a success rate of around 60 to 70% of all cases. This method has had a success rate of 100% of all cases attempted with minimum side effects. It also has specific benefits in haemorrhagic strokes.

This patent relates to the field of pulmonary embolism, coronary and cerebrovascular disease, the most common cause of deaths worldwide. It is estimated that around 50000 deaths occur from coronary heart disease alone every day out of which 60% occur in developing countries. Most of these deaths are due to MI although other causes such as cardiac hypertrophy, arteriosclerosis, aortitis, polyarteritis and other connecting tissue diseases coexist to a minor extent.

Strokes are the third most common cause of death after coronary heart disease and cancer, and 85% of all strokes are of ischemic origin. It is estimated that around 300 of every 100000 people worldwide are affected by strokes, while 60-70 people out of 100000 die due to pulmonary embolism.

In coronary heart disease, sudden deaths from ventricular fibrillation or asystole may occur immediately, and many deaths occur in the first hour. If the patient survives this stage the liability for developing arrhythmias remain. The development of cardiac failure, however, depends on the extent of myocardial damage and may cause death in those who survived the first few hours of infarction. The reduction in oxygen supply (arising from lowered blood supply) leads to reduction in ATP and phosphate levels thereby lowering G-protein transfer to calcium channels and lowered myofilament contractility. Speedy recovery from infarction is vital to survival. Although 95% ATP is produced in the mitochrondia, normal mitochrondial ATP levels may not necessarily mean sufficient ATP concentration in the cytoplasm as cytoplasmic pools of ATP may be depleted despite high mitochrondial ATP levels. (Younss A. M. et. al.; Exploring cardiac biophysical properties, Global Cardiology Science & Practice, gcsp. V 10, p 1 to 12, 2015)

In case of strokes also, rapid reperfusion is necessary. About a fifth of the patients with an acute stroke will die within one month of the event, through repeated cardiovascular/cerebral dysfunction, and those who survive will be left with some physical disability.

Recovery depends on reperfusion and the time it takes. Reperfusion is generally by thrombolysis and the drugs available are streptokinase, alteplase, tenecteplase and reteplase. Here too, time is crucial, and in many cases may lead to death if the reperfusion process is not quick enough to prevent myocardial damage. Tetrahertz therapy by IR and millimetre waves proposed in 2006, using a handheld location imaging probe, did not find much use. (Hoffman, A. K.; pat no 2429158 LOW, GB dated 2 Aug. 2006 and pat no WO/2006/010240 dated Feb. 2, 2006) In case of strokes alteplase and RtPA cannot be used due to risk of haemorrhage, antiplatelet drugs and statins are the mainstay. Anticoagulant and anti-anginal drugs also increase the risk of haemorrhage. Mutations associated with atrioventricular conduction block leading to MI's have been studied (Wang, D. W. et al; Clinical, Genetic and Biophysical Characterization of SCNSA mutations associated with AV conduction block. Circulation, V 105, p 341, (2002). Calcium sensitizers like levosimendan, a new class of drugs that improve altered myofilament contractility have been developed to improve cardiac contraction in a failing heart (gcsp V10 p 1-12 2015, earlier cited in para 0005). Despite all of these, even today the chances of recovery from MI's are around 60% to 70%, and 1 in 4 deaths in the US occur from MI's.

### BRIEF SUMMARY OF THE INVENTION

This invention provides a drug based biophysical method for immediate reperfusion in pulmonary embolism, MI and/or ischemic strokes even when the thrombus/emboli are blocking the arteries at multiple junctures, and does so in the shortest possible time (<5 minutes), thereby minimizing the damage to myocardial/cerebral tissue, and making it possible to treat abnormalities such as ionic alterations as may occur.

### BRIEF DESCRIPTION OF DRAWINGS

FIGS. 1 and 2 refer to the first case study and FIGS. 3 and 4 refer to the second case study: FIG. 1: ECG record of the patient taken about 4 days from the attack. They show ST and T wave abnormalities typical of inferior ischemia.

FIG. 2: ECG record of the same patient taken 24 hrs of the attack. They show T wave abnormalities typical of inferior ischemia.

FIG. 3: MRI scan report of the brain after an ischemic stroke, taken 48 hrs after the stroke. Findings indicate two small acute non haemorrhagic infarcts on the right parietal lobe.

FIG. 4: CT scan report of the brain showing small vessel ischemic changes.

### DETAILED DESCRIPTION OF THE INVENTION

We have developed a drug combination for immediate reopening of blocked arteries, even in cases of multiple site blockages, and which when administered to a patient suffering from pulmonary embolism, MI or an ischemic stroke, will within minutes reperfuse the affected zone. This combination through a method of shear stress, platelet disaggregation and fibrinolysis, fractures and removes the thrombus/emboli within minutes, and reperfuses the pulmonary/myocardial/cerebral infarct area. The signs and clinical features of MI/ischemic strokes are almost immediately removed. Repeat attacks are not normally seen in the next 48 to 72 hrs but if they occur are dealt with similarly. It is used for immediate reperfusion of infarct areas.

We detail two (out of the many) case studies in which the above methods were used.

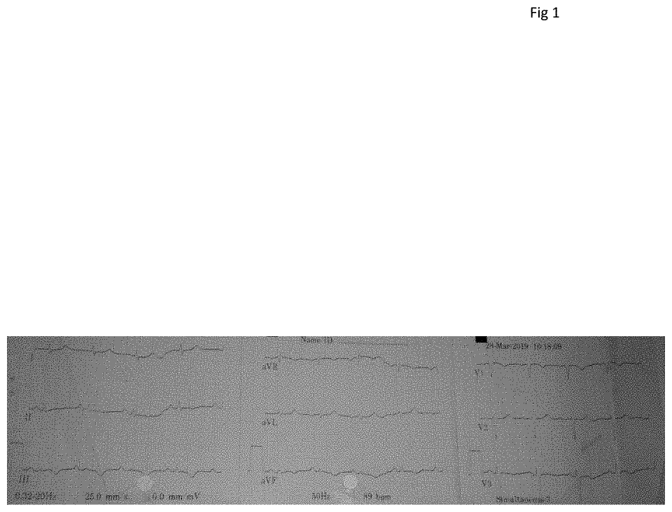

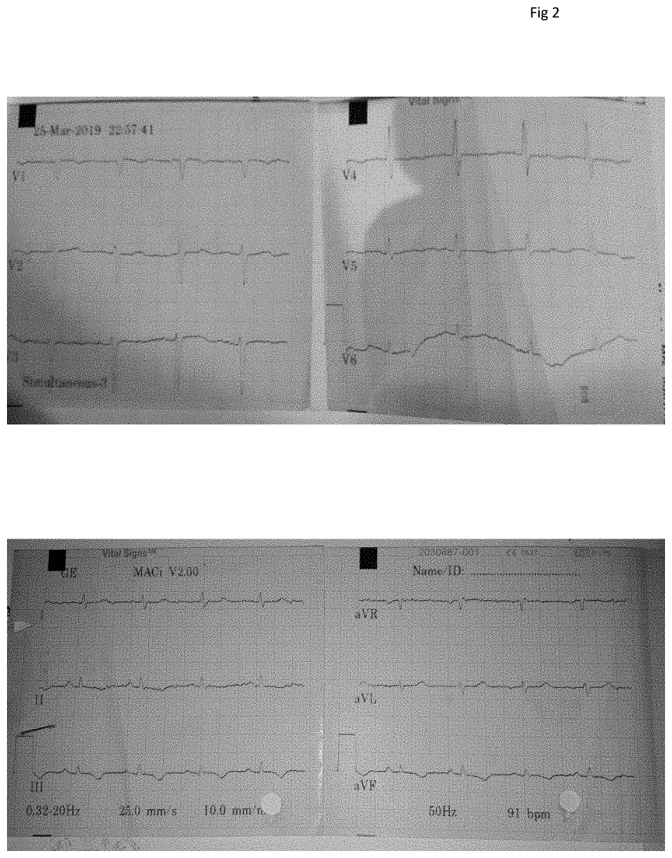

In the first case a 90 years old female patient with type II DM, chronic hypertension, respiratory distress and anaemia was being treated with telmisarten, insulin, isosorbide mononitrate, torasemide, aspirin, atorvastatin, ranolazine, clopidogrel, levosalbutamol aerosol and usual vitamins and steroids. She complained of chest pains, acute dyspnea, vomiting, pallor, sweating, and other symptoms of acute MI. She was treated with the combination and within minutes her chest pain and dyspnea had subsided. The residual fall in pressure was managed and she was sedated and kept on supportive care. Her ECG records, taken within few days after attack, are enclosed as a part of this application (FIGS. 1 and 2). They show characteristic ST and T wave abnormalities. The attack happened at the patient's residence and she was treated there without the requirement of ICU or ICCU facilities. There were no repeat attacks. Her usual drugs were continued in the follow up.

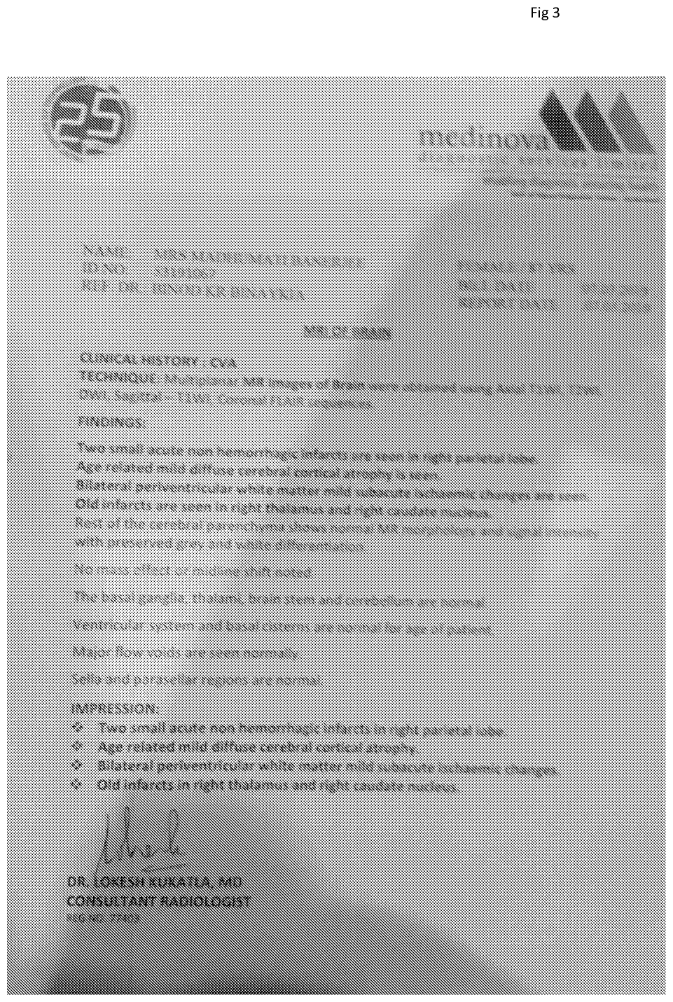

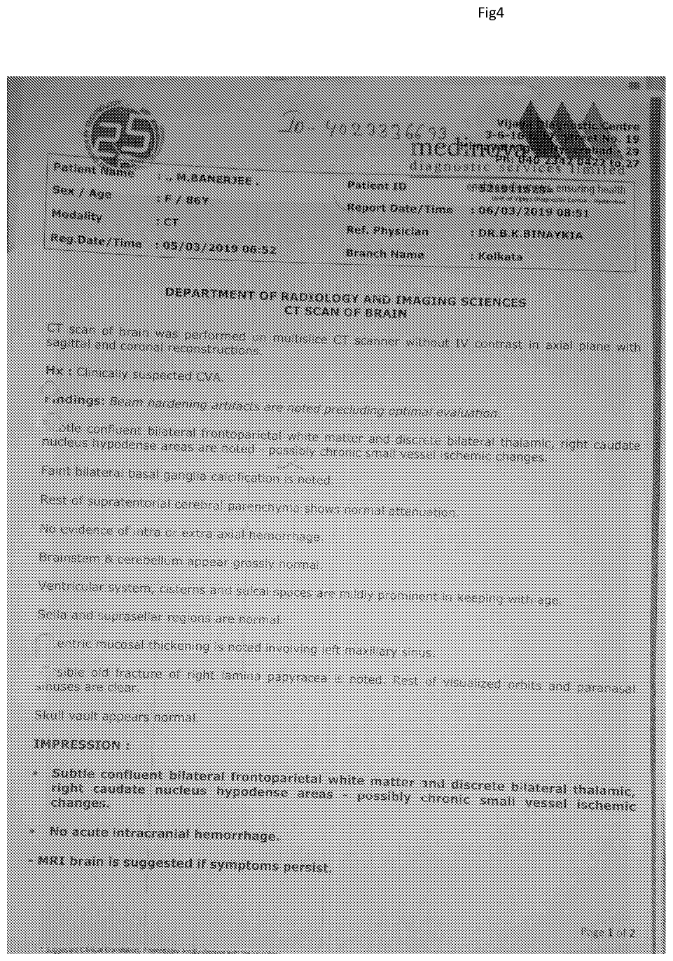

In another case study a female patient aged about 90 yrs suffering from pneumonia, chronic hypertension, type II DM, and anaemia, was being treated with ceftrioxone, telmisarten, torasemide, insulin, isosorbide mononitrate, aspirin, atorvastatin, ranolazine, clopidogrel, oxygen, levosalbutamol aerosol, and vitamins. She refused treatment and suffered a MI with usual symptoms, was reperfused, but still continued to refuse her standard medicines resulting in a second attack and a third one, (all of which were blocked), and finally a cerebral infarction which left her paralysed (not hemiplagia) and unconscious. She was treated with the combination and adrenergics were used to force her heart to function. Her CT and MRI scan showed frontoparietal lobe infarction at two sites. The scan reports are enclosed with this application (FIGS. 3 and 4). She survives and although unable to walk retains her cognitive functions and is able to move her limbs. In the follow up she was
treated with nicorandil, insulin, rabeprazole, rosuvastatin, antianemics and vitamins. There has been no recurrence over the past one year.

None of the above patients had underlying severe valvular disease, or cardiomyopathy, or had pacemakers installed. In both cases drugs were administered when systolic pressure peaked.

The drugs used were chosen from a group of diuretics, adrenergics, antiplatelet agents, statins, CNS stimulants, antihypertensives, vasodilators and steroids used either alone or in combination, as a curative, post occurrence of pulmonary embolism, MI's or ischemic strokes.

### THE BEST WAY OF CARRYING OUT THE INVENTION

The drugs were punched into tablets for oral or sublingual use, and/or nasal solutions made using compatible ingredients, and administered orally, sublingually or nasally. A necessary precaution is that calcium channel blockers should not be used.

Example: Active ingredient—42 mg, lactose—20 mg, PVP—1 mg, ethanol—q.s., talcum—4 mg, magnesium stearate—1.6 mg, starch—10 mg.

The above example is merely an illustration, not exhaustive. The amounts of ingredients may vary depending on active ingredients used.

### INDUSTRIAL APPLICABILITY

This invention would be lifesaving in situations where all existing drugs and treatments fail. We have tried it in innumerable situations, and in extremely critical conditions. In most cases of MI, (including multiple infarcts) the infracted area would be reperfused within minutes blocking the spread of the infarction. In severe cases where the infarct has progressed to acute ventricular failure, it would halt progression, gaining valuable time for corrections to be made to restore cardiac rhythm. In cases of cerebral ischemia, it would stop further infarct spread in cerebral areas almost immediately, and reduce volume of haemorrhage in case of haemorrhagic strokes.

These apart the following applications are significant:
1 ICCU's are welcome, but it can be worked out at patient's homes, gaining time for hospital transfer if required, in critical cases.
2 Ease of administration, no specialised personnel, or equipment, or storage facilities required.
3 It is safe with much lesser incidence of side effects.
4 Cost convenient.
5 Much lesser chances of repeat attacks if the patient is on proper follow up.
6 No risk of hemorrhagic transformation of cerebral infarct and induced fatality, or cerebral haemorrhage post MI treatment, as with TPA or RtPA.
7 Lower risks of post stroke complications, and is 100% successful inpreventing fatalities.

We claim:

1. A drug based biophysical method for the cure of pulmonary embolism, MI's and ischemic strokes, comprising:
A curative drug combination for removal/breaking down emboli blocking arteries, on occurrence of pulmonary embolism or Myocardial infarction or ischemic stroke, by breaking down the points of attachment of the emboli to the vascular walls, followed by fragmentation.
A drug combination for maintaining blood pressure during administration.

2. The method as claimed in claim 1, wherein the drug combination is selected from the group consisting of, diuretics, adrenergics, CNS stimulants, antiplatelet agents, statins, antihypertensives, vasodilators, and steroids, used alone and in combination, for the removal of thromboembolic blockages and reperfusion of arteries gaining precious time to correct ionic imbalances and restore heart rhythm while maintaining blood pressure.
To be noted here, is that none of the ingredients are by themselves thrombolytic, and the novel combination and method causes thrombolysis, when administered post occurrence of MI or pulmonary embolism or ischemic stroke. (The combination is not to be used prophylactically).

3. The method as claimed in claim 1, wherein the improvement is comprised of removal of blockages and reperfusion within few minutes (under 5 min) of oral administration, when compared to approximately 30 minutes or more, taken by currently used drugs (all injectables), leading to fatalities.

4. The product obtained from the method as claimed in claim 1 wherein the improvement comprises of removing all simultaneously occurring embolic blockages and preventing repeated attacks.

5. The product obtained from the method as claim 1, which is the only drug which has been used with 100% success rate in the cure of pulmonary embolism, MI's and ischemic strokes, compared to currently used methods (ATP, RtPA and others) which have an approximately 60% to 70% success rate.

6. The product obtained from the method as claimed in claim 1, which is safer to use, than the currently used products (ATP, RtPA and others), and does not carry the risk of hemorrhagic transformation of cerebral infarcts in patients having cerebral ischemic attacks, as with the currently used products mentioned in claim 5.

7. The product obtained from the method as claimed in claim 1, for use in cure of ischemic strokes, where the improvement is that it can remove thromboembolic blockade of cerebral arteries without resulting cerebral haemorrhage, thereby reducing infarct size, while keeping the heart functioning.

8. The product obtained from method as claimed in claim 1, for use in the cure of haemorrhagic strokes, by reduction of volume of haemorrhage, while keeping the heart functioning.
